(19) United States
(12) Patent Application Publication
(10) Pub. No.: US 2021/0059790 A1
(43) Pub. Date: Mar. 4, 2021

(54) MAXILLARY SKELETAL EXPANDER

(71) Applicant: Yeo Seob LEE, Gwangju (KR)

(72) Inventors: Yeo Seob LEE, Gwangju (KR); Sung Hoon LIM, Gwangju (KR)

(73) Assignee: Yeo Seob LEE, Gwangju (KR)

(21) Appl. No.: 16/689,187

(22) Filed: Nov. 20, 2019

(30) Foreign Application Priority Data
Sep. 3, 2019 (KR) 10-2019-0108993

(51) Int. Cl. A61C 7/10

(52) U.S. Cl. CPC A61C 7/10

(57) ABSTRACT

A maxillary skeletal expander is proposed. The maxillary skeletal expander is composed of a pair of dental arch expansion bodies positioned to face each other in an oral cavity; an expanding screw provided between the pair of dental arch expansion bodies to move the dental arch expansion bodies; a guide provided between the pair of dental arch expansion bodies to safely move the dental arch expansion bodies without deviation; a dental arch expansion body combining means having a fixing piece for penetrating a fixing hole at a selected part in the oral cavity, and configured to be combined to and separated from each of the dental arch expansion bodies.

### CROSS REFERENCE TO RELATED APPLICATION

The present application claims priority to Korean Patent Application No. 10-2019-0108993, filed Sep. 3, 2019, the entire contents of which is incorporated herein for all purposes by this reference.

### BACKGROUND OF THE INVENTION

### Field of the Invention

The present invention relates to a maxillary skeletal expander which may simplify and facilitate a treatment for dental malocclusion, that is, an expansion of maxillary bone or mandibular bone, as well as to reduce pain of a patient and to shorten treatment time.

### Description of the Related Art

A jawbone in an oral cavity is made up of a maxillary bone and a mandibular bone. The upper and lower teeth, respectively connected to the maxillary bone and the mandibular bone, are formed on the upper and lower sides of the oral cavity. Also, the jawbone has a structure in which the upper and lower teeth are interlocked with each other according to a movement of a jaw joint in order to chew food.

In general, the maxillary bone of a normal person is formed to protrude further forward than the mandibular bone. However, sometimes, there may be a case of malocclusion in which the maxillary bone and the mandibular bone are reversely interlocked because the maxillary bone is smaller than the mandibular bone.

When the malocclusion occurs, a corrective treatment is performed to pull the maxillary bone forward and to expand the upper teeth of the maxillary bone to sides.

A maxillary skeletal expander is a device used for orthodontic treatment for malocclusion of a patient whose maxillary bone is less developed than his/her mandibular bone, and the maxillary skeletal expander is provided in order to facilitate the orthodontic treatment by expanding the maxillary bone.

This maxillary skeletal expander has been introduced in Korean Patent No. 10-1723781 (A HYBRID ORTHODONTIC APPLIANCE FOR NON-EXTRACTION ORTHODONTIC TREATMENT) and Korean Patent No. 10-1709164 (MAXILLARY SKELETAL EXPANDER WITH TRIPLE EXPANDING SCREW SYSTEM).

The maxillary skeletal expander according to the related art uses an expanding screw to expand the interval of a main body of the maxillary skeletal expander so that a treatment is performed to make a maxillary arch and a mandibular arch be of the same size. After the size of the maxillary arch and mandibular arch is properly corrected, an orthodontic treatment is performed by attaching brackets to teeth surfaces so that the teeth are positioned evenly.

In addition, recently, a maxillary skeletal expander using a mini screw of Korean Patent Application Publication No. 10-2015-0024245 has been provided as shown in FIG. 1 and FIG. 2, and a configuration of the maxillary skeletal expander is composed of the following: a pair of main bodies 100; an expanding screw 300 provided to adjust the interval between the pair of main bodies; a guide rail 400 having both ends inserted into a pair of main bodies, for guiding movement of the main body; a key hole 500 provided integrally with an expanding screw; a screw insertion part 110 provided integrally with the main body and having a screw insertion hole for guiding a micro screw implanted in an upper jaw; an arm 200 having one end thereof fixed to the main body, and the other end thereof extended toward teeth; a teeth fixing part 600 integrally provided with the arm and fixed to the teeth of the patient.

The maxillary skeletal expander using the mini screw of the configuration described above has a limitation on a range of expanding a dental arch by using the expanding screw 300. Therefore, in order to expand more than the limited range, the entire maxillary skeletal expander using the mini screw had to be replaced. Accordingly, the mini screw S was to be separated from a palate in an oral cavity and a new maxillary skeletal expander was to be fixed in the oral cavity again using the mini screw S, thereby increasing inconvenience and cost of the treatment, aggravating pain of a patient, and delaying the treatment time.

### DOCUMENTS OF RELATED ART

(Patent Document 1) Korean Patent Application Publication No. 10-2015-0024245: MAXILLARY SKELETAL EXPANDER
(Patent Document 2) Korean Patent No. 10-1989351: MAXILLARY SKELETAL EXPANDER HAVING MOVABLE SCREW GUIDE
(Patent Document 3) Korean Patent No. 10-1709164: MAXILLARY SKELETAL EXPANDER WITH TRIPLE EXPANDING SCREW SYSTEM

### SUMMARY OF THE INVENTION

The present invention has been invented to solve the above problems, and its purpose is to provide a maxillary skeletal expander which can ensure that an expansion of a dental arch is performed in a secure, precise, and safe way, and that tooth deformation and damage is prevented. Also, when a further expansion of the dental arch is required after the maxillary skeletal expander of the present invention is fixedly implanted in an oral cavity using fixing pieces to perform the expansion of the dental arch, the maxillary skeletal expander can expand the dental arch by easily and conveniently replacing existing dental arch expansion bodies with a pair of new dental arch expansion bodies having a longer length than that of the existing dental arch expansion bodies, without removing the fixing pieces from positions fixedly implanted in the oral cavity.

In order to achieve the objective of the present invention, there is a technical solution for the problems, including: a pair of dental arch expansion bodies 10 positioned to face each other in an oral cavity; an expanding screw 20 provided between the pair of dental arch expansion bodies to move the dental arch expansion bodies; a guide 30 provided between the pair of dental arch expansion bodies to safely move the dental arch expansion bodies without deviation; and a dental arch expansion body combining means 50 having a fixing piece 56 penetrating a fixing hole 54 at a selected part in the oral cavity, and a combining part 51 combined to and separated from each of the dental arch expansion bodies.

The maxillary skeletal expander of the present invention having a configuration as described above, as mentioned in the above objective, has effects wherein by rotating the expanding screw in a state where the maxillary skeletal expander is fixedly provided in the oral cavity in order to expand the maxillary bones or the mandibular bone, the expansion of the dental arch may be performed in a secure, precise, and safe way, and that tooth deformation and damage may be prevented. Also, when further expansion of the dental arch is required after performing dental arch expansion by fixedly implanting the maxillary skeletal expander of the present invention in the oral cavity using the fixing pieces, the present invention has effects allowing the dental arch to be easily and conveniently expanded to a desired length by replacing the pair of existing dental arch expansion bodies with a pair of new dental arch expansion bodies having a longer length than that of the existing dental arch expansion bodies, without removing the fixing pieces from positions fixedly implanted in the oral cavity, thereby eliminating the inconvenience during the expansion of the dental arch, reducing the pain of a patient, and shortening the treatment time.

### BRIEF DESCRIPTION OF THE DRAWINGS

FIG. 1 is a perspective view of a conventional maxillary skeletal expander.

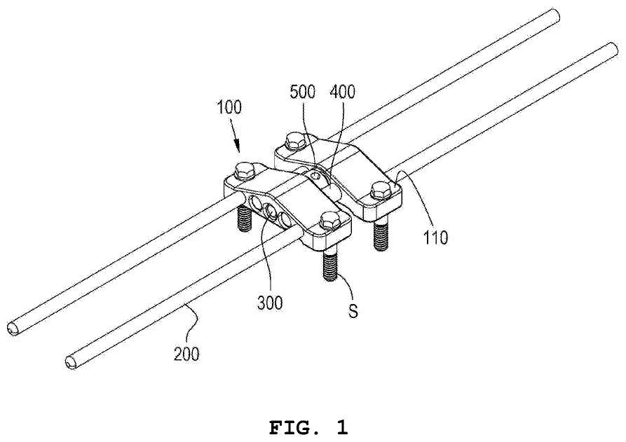

FIG. 2 is an exploded perspective view of the conventional maxillary skeletal expander.

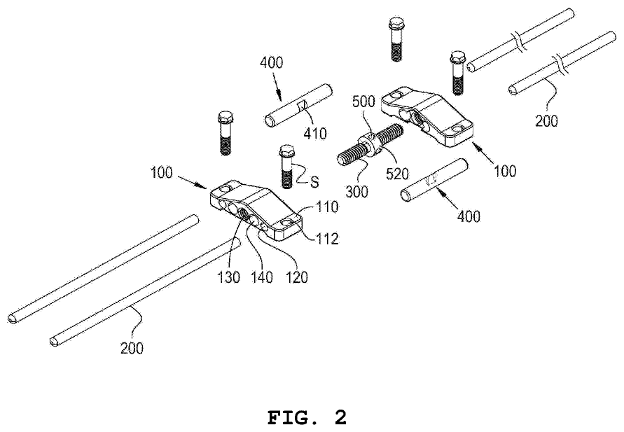

FIG. 3 is an exploded perspective view of the maxillary skeletal expander of the present invention.

FIG. 4 is an assembled perspective view of the maxillary skeletal expander of the present invention.

FIG. 5 is an assembled cross-sectional view of the maxillary skeletal expander of the present invention.

FIG. 6 is a perspective view illustrating an expansion range of the maxillary skeletal expander of the present invention.

FIGS. 7A to 7C are perspective views illustrating an exemplary embodiment of a dental arch expansion body in the maxillary skeletal expander of the present invention.

FIG. 8 is a state diagram illustrating a state of expanding the dental arch using the maxillary skeletal expander of the present invention.

### DETAILED DESCRIPTION OF THE INVENTION

The present invention relates to a maxillary skeletal expander which may simplify and facilitate a treatment for dental malocclusion, that is, an expansion of maxillary bone or mandibular bone, as well as to reduce pain of a patient and to shorten treatment time. An exemplary embodiment of the present invention is described below with reference to the accompanying drawings.

The maxillary skeletal expander A of the present invention is composed of a pair of dental arch expansion bodies 10 positioned to face each other in an oral cavity; an expanding screw 20 provided between the pair of dental arch expansion bodies to move the dental arch expansion body; a guide 30 provided between the pair of dental arch expansion bodies to safely move the dental arch expansion body without deviation; a dental arch expansion body combining means 50 having a fixing piece 56 for penetrating and fixing a fixing hole 54 at a selected position in the oral cavity to be assembled and disassembled from the dental arch expansion body.

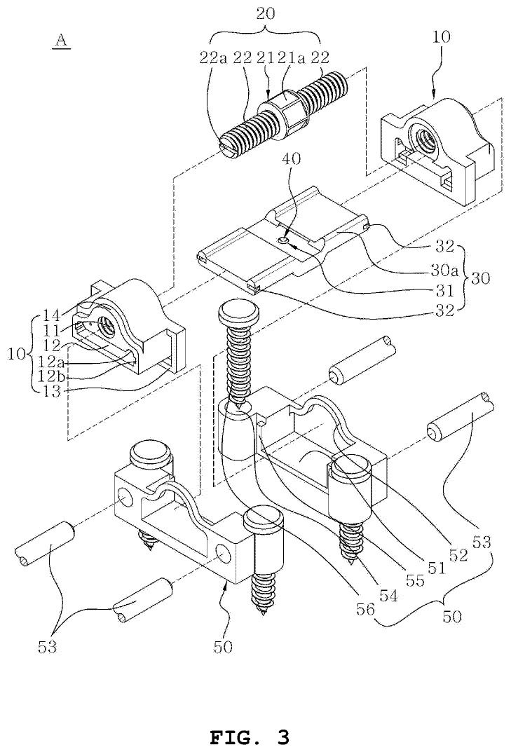

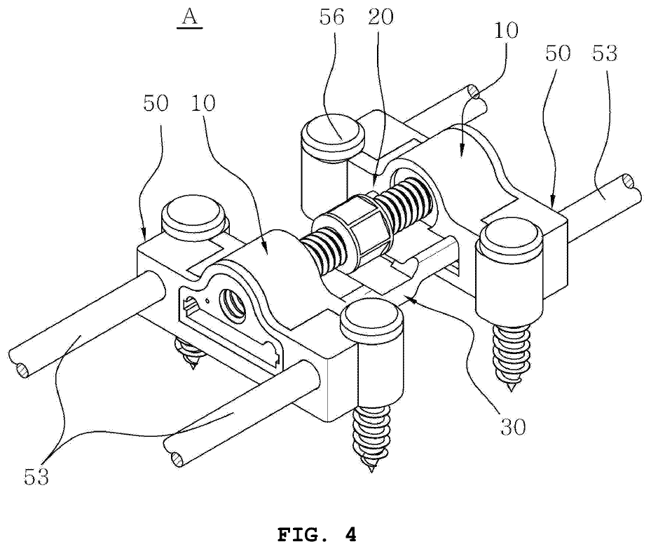

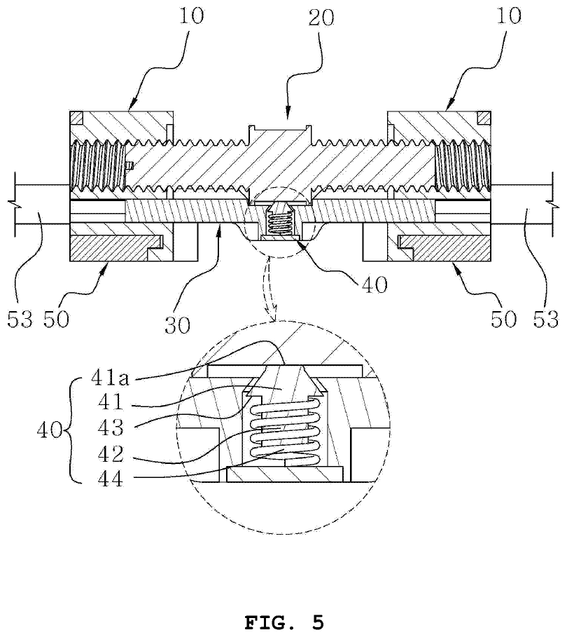

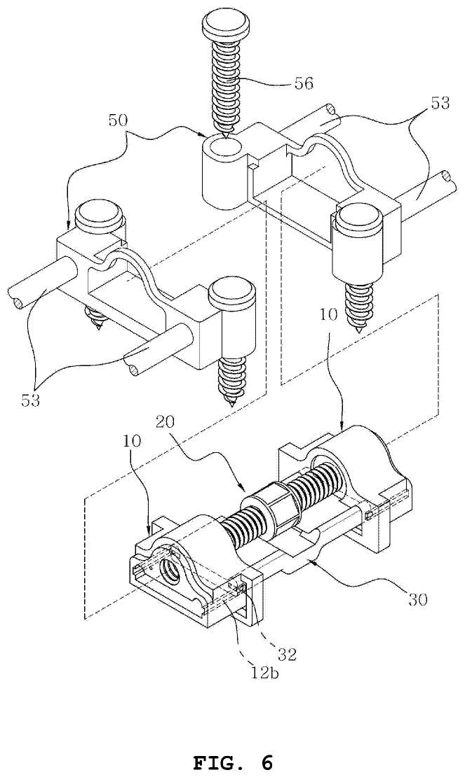

In the maxillary skeletal expander A of the above configuration, a more detailed exemplary embodiment as shown in FIGS. 3 to 7C includes a screw hole 11 provided in the center of the maxillary skeletal expander, and the pair of dental arch expansion bodies 10 provided to face each other in a state where a guide hole 12 is provided at a portion below the screw hole 11.

Then, a rotating body 21 is provided in the center between a pair of dental arch expansion bodies 10, and the expanding screw 20 is provided with bolts 22 on both sides of the rotating body 21.

In addition, the guide 30 is provided to move the pair of dental arch expansion bodies 10 without deviation, in a fitted state coupled to both sides of the guide hole 12 with a flat shape.

Also, the exemplary embodiment includes the dental arch expansion body combining means 50, having a combining part 51, provided to tightly accommodate the dental arch expansion body 10, and provided to separate from and combine to the dental arch expansion body 10 as occasion demands.

The pair of dental arch expansion bodies 10 with the same configuration is provided to face each other on both left and right sides, so that the pair of dental arch expansion bodies 10 moves away or narrows to both the left and right sides according to a rotation direction of the expanding screw 20.

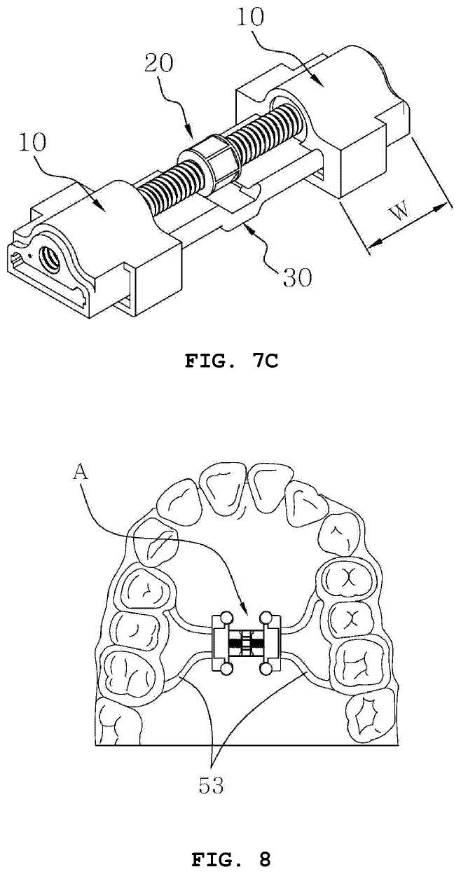

As described above, the dental arch expansion body 10 is widened to the left and right sides in order to expand the maxillary bone or mandibular bone, as shown in FIG. 8. Since the both sides of the guide 30 having a shape conforming to the guide hole are coupled to the guide hole 12 with a flat shape in the expansion process, the expansion is performed in a stable and solid movement.

In addition, in the present invention, the both sides of the guide 30 coupled to the guide hole 12 are also provided with a flat shape so as to be tightly coupled to and conforming to the guide hole 12. Thus, it is effective that the movement is precise and stable without deviation and without moving from left to right of the dental arch expansion body 10.

In addition, the expansion of the dental arch expansion body 10 is performed with a rotation of the expanding screw 20 which is positioned between the pair of dental arch expansion bodies 10, and a rotating body 21 is provided in the center thereof. Also, the bolts 22 are provided on both sides of the rotating body 21 so that the bolts 22 on both sides are screwed into the respective screw holes 11.

Meanwhile, a configuration of the exemplary embodiment of the rotating body 21 is composed of a polygon provided with at least five or more flat surface parts 21a connected to each other along the outer circumferential surface, so that the expanding screw may be rotated by using a polygonal wrench.

The reason why the rotating body 21 is provided into a polygonal shape in which five or more flat surface parts 21a are connected to each other is that a vertical end part of a stopper 40 supports a flat surface part 21a to control the rotation of the expanding screw 20 after completing the expansion of the dental arch expansion body 10.

Since the dental arch expansion body 10 is pressured at various angles in a fixed state after the expansion, the expanding screw 20 may be rotated by pressure to allow the dental arch expansion body 10 to move from a fixed position. Thus, a jawbone expansion may have a different result from the intended purpose. Accordingly, the stopper 40 may be provided in the center of the guide 30 to support the expanding screw 20 to be firmly fixed.

In an exemplary embodiment of the stopper 40 performing a function as described above, the stopper includes: a top part 41 having a flat surface 41a and composed in a form of narrow upper side and wide lower side; a fitting part 42 extending from the lower part of a holder part 41 and seated in a seating groove of a stopper combining part 31; an elastic spring 44 provided between the stopper combining part 31, which is a seating groove, and the fitting part 42; and an end step 43 preventing separation of the elastic spring 44 provided at a boundary part between the holder part 41 and the fitting part 42.

The reason why the stopper 40 of the above-described configuration is provided is that the elastic spring 44 pushes up the end step 43 so that the flat surface 41a of the holder part 41 is tightly in contact with the flat surface part 21a of the rotating body 21 to firmly support the expanding screw 20, thereby controlling the rotation of the expanding screw 20.

In addition, the dental arch expansion body combining means 50 of the present invention is provided with the combining part 51 provided to tightly accommodate the dental arch expansion body 10 and provided to separate from and combine to the dental arch expansion body 10 as occasion demands.

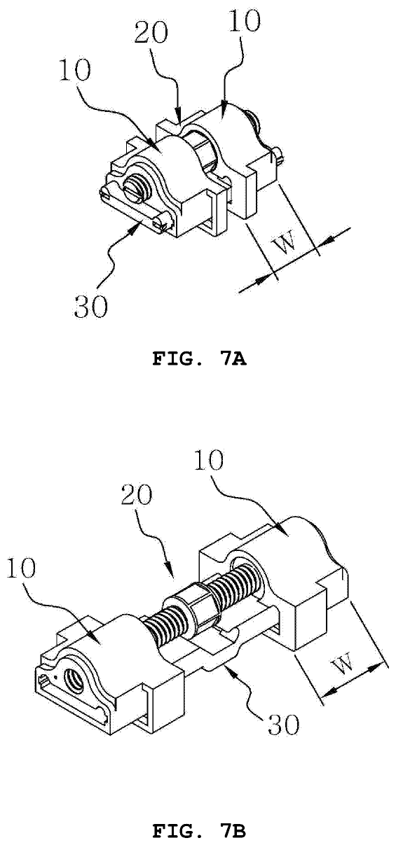

The reason for providing the dental arch expansion body combining means 50 of the present invention as described above is that since the length of the dental arch expansion body 10 is provided to have a variety of sizes as shown in FIGS. 7A to 7C, depending on the extent of the dental arch expansion, since the length W is provided to have a variety of sizes depending on the degree of the dental arch expansion, it is intended to be used while replacing the dental arch expansion body 10 provided with such a variety of sizes.

The length W of the dental arch expansion body 10 is different because sizes of the oral cavity is different from person to person, so that the size of the dental arch expansion body may be selectively applied by individual people and the degree of expansion of a jawbone may be selectively adjusted for individual needs.

In addition, the dental arch expansion body 10 as described above is used because when further expansion of the dental arch is required after performing dental arch expansion by fixedly implanting the maxillary skeletal expander of the present invention in the oral cavity using the fixing pieces 56, the maxillary skeletal expander of the present invention allows the dental arch to be easily and conveniently expanded by replacing the existing dental arch expansion bodies with a pair of new dental arch expansion bodies having a longer length than that of the existing dental arch expansion bodies W, as shown FIGS. 7A to 7C, without removing the fixing pieces 56 from positions fixedly implanted in the oral cavity.

An exemplary embodiment of the dental arch expansion body combining means 50 of the present invention is composed of the fixing hole 54 for fixing the fixing piece 56 on both sides, a support band 52 supporting the rear upper part of the dental arch expansion body 10 at the rear upper part, and a fixing arm 53 provided on both rear sides.

In the dental arch expansion body combining means 50 of the above-described configuration, the fixing hole 54 is a position for being fixed to the jawbone using the fixing piece 56 in a state where the dental arch expansion body 10 is adjusted, the support band 52 is to fixedly support the dental arch expansion body 10 so that dental arch expansion body 10 is not to be moved, and the fixing arm 53 is to be fixed on the teeth.

Meanwhile, the fixing arm 53 is not mandatory but is optional, and fixing both the dental arch expansion body combining means 50 and the dental arch expansion body 10 is performed by being fixed to the jawbone by fitting the fixing pieces into the fixing hole 54.

Also, the reason why recessed groove 12a is provided at both sides of the guide hole 12, and protrusion 30a coupled to the recessed groove 12a is provided at both upper parts of the guide 30 is to make the dental arch expansion body 10 to move more safely when moving along the guide 30.

In addition, the dental arch expansion body 10 is composed of a combining groove 13 continuously formed at both the lower part and front both sides of the dental arch expansion body, and a combining end step 14 provided in the rear upper part. The reason why the combining end step 14 is so provided is to fixedly support dental arch expansion body 10 by being tightly coupled to the support band 52. Also, the reason why the combining groove 13 is so provided is that the combining protrusion 55 to be described below is tightly coupled by inserting into the combining groove 13 so as to make a firm coupling between the dental arch expansion body 10 and the dental arch expansion body combining means 50.

In addition, the reason why the center part of the guide 30 is so provided with the stopper combining part 31 having the seating groove for seating a lower part of the stopper 40 is to make the stopper 40 to be operated smoothly.

In addition, a key groove 12b provided on side surfaces of the guide hole 12 limits an amount of movement when the pair of dental arch expansion bodies 10 are moved by coupling to a key 32 provided in the guide 30. To this end, when the key 32 is positioned at the end part of the key groove 12b, the key 32 is prevented from being moved any further so as to keep the guide 30 from departing from the dental arch expansion body 10.

As described above, the maxillary skeletal expander of the present invention, as described in the above effects, has merits that the expansion of the dental arch may be performed in a secure, precise, and safe way, and that tooth deformation and damage may be prevented, without the pair of dental arch expansion bodies being removed, in a state where the maxillary skeletal expander is fixedly provided in the oral cavity in order to expand the maxillary bones or the mandibular bone in any case. Also, when further expansion of the dental arch is required after performing the dental arch expansion by fixedly implanting the maxillary skeletal expander of the present invention in the oral cavity using the fixing pieces, the present invention has effects allowing the dental arch to be easily and conveniently expanded by replacing existing dental arch expansion bodies with a pair of new dental arch expansion bodies having a longer length than that of the existing dental arch expansion bodies, without removing the fixing pieces from positions fixedly implanted in the oral cavity, thereby eliminating the inconvenience during the expansion of the dental arch, reducing the pain of the patient, and shortening the treatment time.

What is claimed is:

1. A maxillary skeletal expander comprising:
a pair of dental arch expansion bodies (10) positioned to face each other in an oral cavity;
an expanding screw (20) provided between the pair of dental arch expansion bodies to move the dental arch expansion bodies;
a guide (30) provided between the pair of dental arch expansion bodies to safely move the dental arch expansion bodies without deviation; and
a dental arch expansion body combining means (50) having a fixing hole (54) penetrated by a fixing piece (56) at a selected part in the oral cavity and a combining part (51) combined to and separated from each of the dental arch expansion bodies.

2. The maxillary skeletal expander of claim 1, wherein each of the dental arch expansion bodies (10) comprises:
a screw hole (11) provided in a center thereof;
a guide hole (12) provided at a portion below the screw hole;
a recessed groove (12a) and a key groove (12b) respectively provided at each of both sides of an upper part of the guide hole (12) and at each of both sides of the guide hole (12);
a combining groove (13) continuously formed at front both sides and a lower part thereof; and
a combining end step (14) provided in a rear upper part thereof.

3. The maxillary skeletal expander of claim 1, wherein the expanding screw (20) is positioned between the pair of dental arch expansion bodies, and comprises:
a rotating body (21) having a polygonal shape with at least five or more flat surface parts (21a) connected to each other along an outer circumferential surface of the rotating body (21); and
a bolt (22) provided on each of both sides of the rotating body (21).

4. The maxillary skeletal expander of claim 1, wherein the guide (30) is provided with a flat shape conforming to the guide hole (12), and comprises:
a stopper combining part (31) provided in a center thereof and having a flat shape, where a stopper (40) is installed; and
a protrusion (30a) and a key (32) respectively coupled to a recessed groove (12a) and a key groove (12b) that are respectively provided at each of both sides of an upper part of the guide (30) and each of both sides of the guide (30).

5. The maxillary skeletal expander of claim 4, wherein the stopper (40) for controlling movement of the rotating body (21) is provided in a central part of the guide (30), and comprises:
a holder part (41) composed in a form of narrow upper side and wide lower side, and having a flat surface (41a) at a top thereof;
a fitting part (42) extending from a lower part of the holder part and seated in a seating groove of the stopper combining part (31);
an elastic spring (44) provided between the stopper combining part (31) and the fitting part (42); and
an end step (43) provided at a boundary part between the holder part (41) and the fitting part (42) and configured to prevent separation of the elastic spring (44).

6. The maxillary skeletal expander of claim 1, wherein the dental arch expansion body combining means (50) is provided at each of both sides thereof with the fixing hole (54) for fixing the fixing piece (56), and comprises a support band (52) that is provided at a rear upper part thereof to support a rear upper part of each of the dental arch expansion bodies.

7. The maxillary skeletal expander of claim 1, wherein
a fixing arm (53) is provided at the rear of the dental arch expansion body combining means (50), and
a combining protrusion (55) is provided at the front of the combining part (51) to be coupled, by insertion, with the combining groove (13).
